(19) United States
(12) Patent Application Publication
(10) Pub. No.: US 2020/0294619 A1
(43) Pub. Date: Sep. 17, 2020

(54) METHOD FOR COMPACT NOMENCLATURE FOR DNA SEQUENCES

(71) Applicant: NicheVision, Inc., Akron, OH (US)

(72) Inventors: Brian A. Young, Port Orange, FL (US); Tom A. Faris, Akron, OH (US); Luigi Armogida, Medina, OH (US)

(73) Assignee: NicheVision, Inc., Akron, OH (US)

(21) Appl. No.: 16/725,697

(22) Filed: Dec. 23, 2019

Related U.S. Application Data
(60) Provisional application No. 62/785,040, filed on Dec. 26, 2018.

(51) Int. Cl. G16B 20/20; C12Q 1/6809; C12Q 1/6869; G06F 3/048; G06F 16/22; G06F 16/28; G06F 17/16; G16B 30/10; G16B 30/20; G16B 50/50; G16B 50/40; G16B 50/30

(52) U.S. Cl. CPC G16B 20/20; C12Q 1/6809; C12Q 1/6869; G06F 3/048; G06F 16/2282; G16B 50/30; G06F 17/16; G16B 30/10; G16B 30/20; G16B 50/50; G16B 50/40; G06F 16/285

(57) ABSTRACT

A computer-implemented method of performing forensic analysis of DNA sequences includes computing a digest of a raw DNA sequence, where the digest is a numerical value which can be in a base-16 numeral system. The digest numerical value is converted into a converted numerical value which can be a base-26 numeral system, selected to produce a label consisting of letters that can be allocated incrementally to produce labels with the minimum number of letters necessary to avoid duplicate labels for different DNA sequences within a given domain of sequences. Distinct compact labels for distinct DNA sequences are useful in computer interfaces, verbal communications, comparing DNA sequences of different individuals, expressing relationships between sequences, and other situations where compactness is desirable.

### REFERENCE TO AN ELECTRONIC SEQUENCE LISTING

The contents of the electronic sequence listing (SequenceListing-42280_50004.txt; Size: 6,306 bytes; and Date of Creation: May 14, 2020) is herein incorporated by reference in its entirety.

This application claims the benefit of U.S. Provisional Application No. 62/785,040, entitled. METHOD FOR COMPACT NOMENCLATURE FOR DNA SEQUENCES, filed Dec. 26, 2018, which is fully incorporated herein by reference.

### I. BACKGROUND OF THE INVENTION

### A. Field of Invention

This invention pertains to the field of nomenclature for nucleic acid sequences. In particular, the invention pertains to a system and method for generating a nomenclature for DNA for use in a method of forensic DNA analysis. More specifically, it relates to a process for generating highly compact distinct labels for complex DNA sequences suitable for display in computer software, and suitable for vocal and written descriptions of sequences between practitioners or in jurisprudence settings, or with general audiences. The nomenclature method is independent of any external reference. The nomenclature method is robust to the length of DNA sequences and generates a distinct compact label for any DNA sequence including those that have not been observed previously. The method is robust to any class of genetic marker including short tandem repeats (STR), single nucleotide polymorphisms (SNP) deletion/insertion polymorphisms (DIP), haplotypes, and microhaplotypes. In other embodiments the invention relates to a system and method for expressing the relationship between the original true sequences and DNA sequences that arise as artifacts of laboratory analysis methods.

### B. Description of the Related Art

It is well known that the DNA molecule represents the genetic heredity of an organism. DNA is a molecule formed as a “double helix” of two polymeric strands comprised of monomeric units consisting of nucleotides. Each nucleotide consists of a deoxyribose sugar unit, a phosphate unit and one of four nucleobases adenine, guanine, cytosine, and thymine. DNA sequences refer to the specific order of the nucleotides on a DNA strand. DNA sequencing methods determine the order of nucleotides in a DNA strand segment and report the results as a sequencer read (aka read). Reads are string representations of DNA sequences. Consequently, DNA sequencing has a myriad of applications in biological research and medical diagnosis.

DNA sequencing methods determine the order of nucleotides in a DNA strand segment and report the results as a sequencer read (aka read), Reads are string representations of the DNA sequences in which each nucleotide is represented by a single letter. DNA sequencing has specific applications in forensic analysis, DNA sequences can be used to infer physical appearance traits, relatedness between individuals, and regions of ancestral origin of individuals. One specific application is for identification of suspects in police investigations. DNA in a sample of human tissue or fluid taken from a crime scene that can be matched with DNA in a different sample taken directly from a suspect or other person of interest to positively identify that individual at the crime scene.

It is known in the art to use shorthand terminologies when referring to nucleic acid molecules such as DNA and RNA. These terminologies are collectively referred to as nucleic acid nomenclature. The most common nomenclature is to represent the nucleotide bases in a base pair as letters—for example, an adenine nucleotide is abbreviated as A, guanine as Ci, cytosine as C, and thymine as T. DNA sequences are commonly represented as a string of these letters. The letters can be either upper case or lower case. Additional letters are used to indicate ambiguity in the true identity of the nucleotide. For details on ambiguity codes, see H.B.F. Dixon, H. Bielka, C. R. Cantor, Nomenclature committee for the International Union of Biochemistry (NC-IUB). Nomenclature for incompletely specified bases in nucleic acid sequences. Recommendations 1984, Proc. Natl. Acad. Sci. U.S.A. 83 (1986) 4-8. doi:10.1073/pnas.83.1.4.

DNA sequences can be very long. The simple nomenclature of representing DNA sequences as a long string of letters can result in long DNA sequences of hundreds of characters that are difficult to display in DNA analysis software systems. Such long strings of letters can be especially difficult for humans to read and it can thus be hard to compare one sequence to another or to describe in written or verbal communication.

Compact expression of DNA sequences is a well-recognized problem in the fields of forensic DNA analysis and medical genetics. Nomenclature methods currently used in forensics are either: not compact; or are not a complete solution in the sense that they are not robust to novel or previously unobserved sequences or depend upon external references; or require look-up tables that must be separately maintained. In other words, current methods of presenting DNA sequence nomenclature either depend upon the external references (to compact the nomenclature), which must be updated each time the external reference is changed or are not compact enough for easy use in computer displays.

The nomenclature method of Parsons et al., described by the DNA Commission of the International Society for Forensic Genetics (ISFG), produces a verbose description of DNA sequences that includes genomic coordinates in genome assemblies published by the Genome Reference Consortium (GRC). The GRC genorne assembly is periodically updated meaning that the ISFG nomenclature has an external dependency and is not self-contained. Additionally, the ISFG nomenclature is not compact, and typically includes about 75-100 characters rendering it too long for display in many software interfaces. Additionally, the ISM nomenclature is too verbose for easy vocalization. Additionally, the ISFG nomenclature is described specifically for STR markers and is not robust to other classes of genetic marker. (For a description of the ISFG nomenclature see Parsons, W., et al. Forensic Sci. Intl. Genet. 22 (2016) 54-63. doi:10.10161.fsigen.2016.01.009 and. Phillips, C., et al., Forensic Sci. Intl. Genet. 34 (2018) 162-169. doi:10.1016/fsigen.2018.02.017.)

Another nomenclature method of van der Gaag specific to STR. markers represents the DNA sequence by specifying the number of times tandemly repeated DNA motifs in the DNA segment are repeated. In this nomenclature, either the repeated motifs, or the index numbers indicating the number of repeats of the motif are enclosed in brackets. (For a description of one indexed bracket nomenclature see van der Gaag, K. J., Forensic Sci. Int. Genet. Suppl. Ser. 5 (2015) e542-e544. doi:10.1016/j.fsigss.2015.09.214.)

Another nomenclature method of Just et al. specific to STR markers represents the DNA sequence using only numbers that represent the total number of tandemly repeated. motifs in a sequence and the number of tandem repeats in the motif with the longest uninterrupted stretch (LUS). These two numbers are separated by an underscore. In simple repeat STR loci, the LUS is equal to the total number of repeats. This nomenclature is specific for SIR markers and is not able to account for sequence variation such as SNP variation in an STR locus or in its nearby flanking regions that does not involve the number of tandem repeats. (For a description of this nomenclature see Just, R. S. and Irwin, J. A., Forensic Sci. Int. Genet. 34 (2018) 197-205. cloi:10.1016/j.fsigen.2018.02.016.)

Another nomenclature method of Van Neste is key-value database-managed. systems where compact keys point to distinct DNA sequence values in the database or in a separate database. A shortcoming of database systems is that novel sequences not previously observed will not be in the database. Moreover, database systems require regular curation. (For a description of the key-value database system see Van Neste, C., Forensic Sci. Int. Genet. 20 (2016) e1-e3. doi :10.1016/j.fsigen.2015.09.006.)

A nomenclature of Kidd has been proposed for microhaplotype markers intended for forensic DNA analysis. In this system, a string of characters designates the genomic location of the microhaplotype and a different string of characters identify the individual haplotypes possible at the location. The locus identifier consists of a string of characters of which the first 2 are “mh” followed by 2 characters indicating the chromosome number, followed by 2-4 characters assigned by the laboratory that first discovers or characterizes the site, followed by an indeterminate number of characters assigned by the discovering laboratory to be used to discriminate different microhaplotypes identified by the same laboratory, but located on the same chromosome. (For a description of this nomenclature, see Kidd, K. K., Human Genomics (2016) 10:16 DOI 10.1186/s40246-016-0078-y.)

The individual haplotypes at a locus are indicated by a concatenation of the letters indicating the state (aka allele) of each SNP marker within the multi-SNP microhaplotype. For example, the microhaplotype mh02KK-003 consists of the three SNP loci rs260694 (a G/T SNP), rs11123719 (a T/C SNP), and rs11691107 (a C/T SNP), and one possible haplotype version (aka allele) is indicated by ‘GCC’. A drawback of this nomenclature system is that it does not account for the possible observations of additional rare minor allele SNPs within the previously described microhaplotype by operational forensic laboratories who are unlikely to formally publish the discovery.

In computer science, mapping functions are procedures that map arbitrarily large data items to a much smaller bit string that for all practical purposes uniquely identifies the original data item. A wide range of variants exist including checksum functions, fingerprints, cyclic redundancy checks, hash functions, and cryptographic hash functions. One specific class of cryptographic hash function is the secure hash algorithm (SHA) family of algorithms. These are one-way functions in which each input string produces a specific output string (aka message digest or hash value). The implication of this is that the same hash value is always generated for the same input string.

The present invention provides a method of compacting a nomenclature by producing a label or code in the form of a short string of characters that is easy to read and vocalize, is robust to any type of genetic marker, and any sequence regardless of whether it has been observed previously, and is useful for further additional processing, particularly for DNA forensic analysis.

### II. SUMMARY OF THE INVENTION

The invention pertains to a method and system for generating a practical nomenclature for DNA sequences. A DNA sequence typically is represented by a continuous string of A, T, C and G letters. The letters of DNA sequences may be in upper case or lower case or mixed case. DNA sequences processed by the method of this invention should be in consistent case, preferably upper case, so that the labels produced by the method are repeatable thereby facilitating sequence comparisons. The method operates on the ASCII (American Standard Code for Information Interchange) binary representation of characters. The lower case “a” character is represented by binary “1100001”; or hexadecimal “61” whereas the upper case “A” character is represented by binary “1000001” or hexadecimal “41”. Thus, the method produces distinctly different labels for English alphabet characters of different case.

The DNA sequence is processed in several steps. A) A hash function such as the SHA-256 function is used to produce a fixed-length bit string termed a message digest. The SHA-256 hash function produces a 256-bit fixed-length bit string. Other hash and related functions produce different fixed-length bit strings. Message digests are typically expressed in the hexadecimal numeral system where digits are represented by the set of characters {0 . . . 9; a . . . f}. B) The hexadecimal hash value is converted to a hexavigesimal (base-26) value where digits are represented by the characters {0 . . . 9; a . . . p}. Hash value letter characters may be represented in lower or upper case depending upon the hash algorithm implementation. C) If present in lower case, hash value letters are converted to upper case. D) Individual hexavigesimal digits are converted to their ASCII (decimal) equivalent. E) ASCII values are differentially offset depending upon the character class in step C. If the character was a letter, the ASCII value is incremented by 10 (decimal) and if the character was a number the ASCII value is incremented by 17 (decimal). The purpose of this differential offsetting is to lead to labels produced by the method that consist solely of capital English letters. F) The ASCII values are converted back to ASCII characters. Due to the offset step (E) the resulting characters are all capital English letters {A . . . Z}. G) The letters of the final digest are reversed such that the hexavigesimal value represented by the letters becomes little-endian. The reversal step is preferably performed as the last step prior to dynamic allocation of letters but may optionally be performed at any step of the method without affecting the resulting labels. H) Letters in the final digest are dynamically allocated to provide the minimum number of letters required to resolve collisions defined as where a distinct label corresponds to more than one distinct DNA sequence. The nomenclature described in this method can be optional appended to other nomenclature such as allele number nomenclature to provide cross compatibility. Taking the example in FIG. 1, the method generates a ‘KL’ nomenclature which combined with the D2S441 STR locus allele number 12 nomenclature could optionally be described as a 12 KL at D2S441.

Each significant digit of the converted numerical value is associated with a respective alphanumeric character of an encoding system, which can be ASCII. Each of the respective alphanumeric characters are translated into their corresponding unique numbers of the ASCII encoding system. The corresponding unique numbers are processed into the end product represented by the code of respective alphabetic characters, where the code has the desired number of characters. In this manner, the code represents a compact nomenclature of the raw DNA sequence.

According to one aspect of the present invention, the method of compacting a nomenclature converts DNA sequences to a short letter-based code that is much easier to read and to display on a computer monitor than the original sequence, and more readily useful for rapid processing on computer systems, and easier to communicate in written or verbal form.

According to another aspect of the present invention, the method of compacting a nomenclature is deterministic since a given DNA sequence always coverts to the same letter-based code.

According to still another aspect of the present invention, the nomenclature is self-contained and does not depend upon any external references, unlike the prior art nomenclature methods.

According to still another aspect of the present invention, the nomenclature is compact and typically requires less than 10 characters regardless of the length or complexity of the source DNA sequence from which it is taken.

According to further aspect of the present invention, the method of compacting a nomenclature is especially useful in forensic analysis in which numerous DNA sequences from each individual are considered, and DNA sequences from one person are compared to DNA sequences of other persons.

According to still another further aspect of the present invention, the method of compacting a nomenclature is useful for concisely indicating relationships between correct sequences and artifactual sequences arising from DNA analysis errors.

According to yet another further aspect of the present invention, the method of compacting a nomenclature is especially useful to police caseworkers who require quick and accurate comparisons of different DNA sequences without resorting to complex string comparison methods such as sequence alignment.

Still other benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, the embodiments of which will be described in detail in this specification and illustrated in the accompanying drawings which form a part hereof and wherein:

FIG. 1 is a schematic view of an exemplary DNA sequence (SEQ ID NO. 26) processed in accordance with the present invention;

FIG. 2 is a flow chart according to an exemplary embodiment of the present invention; and

FIG. 3 is a schematic view depicting the processing performed in the flow chart of FIG. 2, according to an exemplary embodiment of the present invention (SEQ ID NO. 26).

### IV. DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the present invention.

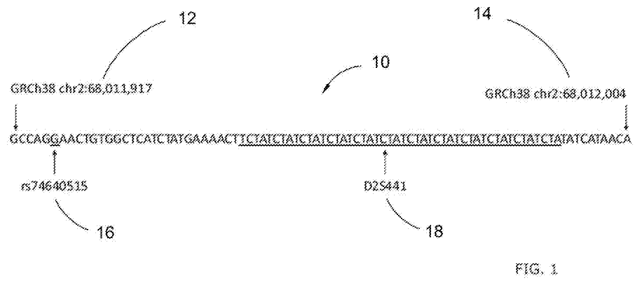

Referring now to the drawings wherein the showings are for purposes of illustrating embodiments of the invention only and not for purposes of limiting the same, FIG. 1 depicts a typical DNA sequence 10 processed in accordance with the computer-implemented method of the present invention. The locus 12 is the location on a chromosome that marks the beginning of the DNA sequence 10. Another locus 14 is the location that marks the end of the DNA sequence 10. An SNP 16 (single nucleotide polymorphism) is a nucleotide position or location that varies (aka is polymorphic) among individuals of a population. Thus, different nucleotides may be present at that location in different individuals.

An STR 18 (short tandem repeat) is a segment of the DNA sequence 10 in which certain DNA “motifs” are tandemly repeated a certain number of times. In the indicated STR 18, the motif is defined by the bases TCTA repeated twelve times, or (TCTA)₁₂. Other STRs are defined by different bases and different numbers of repeat units. For a given DNA sequence 10, one individual can have an STR 18 with a certain number of repeat units, while a different individual can have a different number of repeat units in the same DNA sequence 10. These different forms of the STR locus 18 are termed alleles. Additionally, different individuals may have different nucleotides at SNP 16.

Different STRs having different numbers of repeat units can occur in different DNA sequences. The frequencies of the various alleles within a population can be determined and therefore the rarity of a multi-locus allelic profile can be calculated from the population frequencies. The odds of any two individuals having the same numbers of repeat units in each locus of a multilocus profile can be sufficiently small that such specific patterns of STRs can serve as a “fingerprint” of each individual. Thus, STRs in DNA sequences and can be used in forensic analysis to identify a suspect from DNA in a sample left at a crime scene.

In the current, existing forensic analysis technology used by law enforcement agencies in many countries including the US FBI, STR alleles are reported in a compact nomenclature termed allele numbers, which reflect the length of STR alleles, but not the sequence. Taking the example in FIG. 1, the D2S441 STR locus exhibits the allele number 12. This method cannot discriminate between alleles that exhibit the same length, but different sequences. For example, the repeat structure of STR 18 in FIG. 1 can be displayed in indexed bracket notation as [TCTA]12, which corresponds to the STR sequence present in GenBank accession MH167325.1. However, the STR 18 sequence may differ at a single base such that the indexed bracket notation is “[TCTA]10 TCTG [TCTA]”, which corresponds to the STR sequence present in the GenBank accession MI1167326.1. When expressed in the common allele number notation, these two STR alleles cannot be discriminated.

Further, this current, existing method cannot discriminate alleles that exhibit DNA sequence variation at an SNP locus that is nearby to an STR locus, and therefore present in the same sequencer read string. Taking the example in FIG. 1, the SNP location 16 (SNP name rs74640515) may exhibit either an A nucleotide or a G nucleotide on the GenBank top strand in different persons. Thus, two persons may exhibit the same allele number at STR 18, but different nucleotides (aka alleles) at SNP position 16. The allele number nomenclature will not discriminate these two sequences. However, consideration of the entire sequence between positions 12 and 14 can be used to discriminate the two individual sequences. The present method provides a short nomenclature for use in discriminating two read sequences when they differ by sequence, including when they differ by length, which by definition is also a difference in sequence.

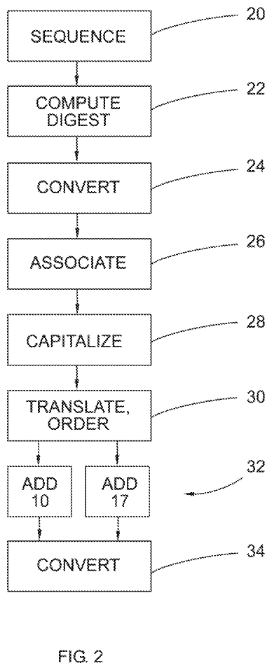

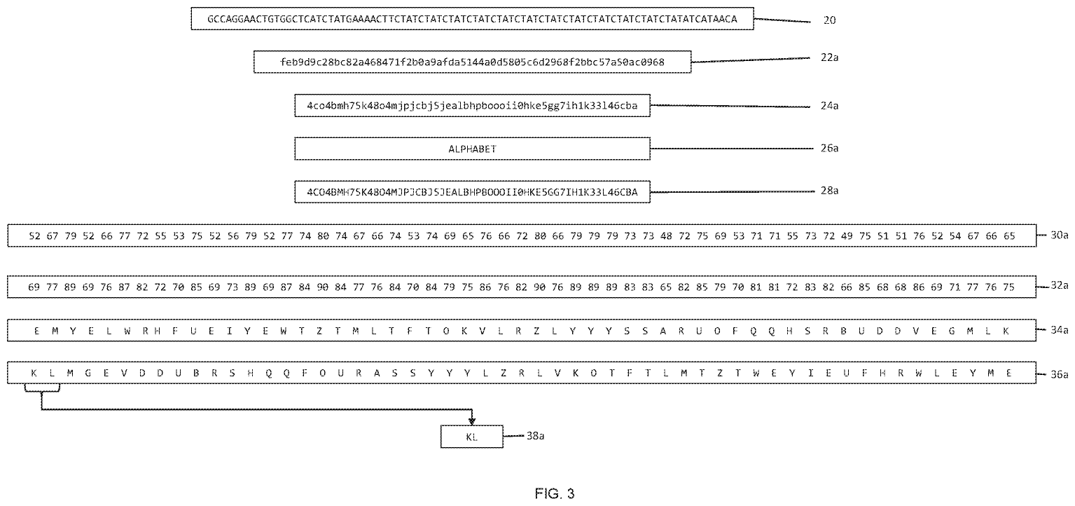

Referring now to FIGS. 2 and 3, the present invention includes a method for distinctly labeling the entire DNA sequence 10 in a compact nomenclature. FIG. 2 is a flow chart showing the general methodology of an exemplary embodiment of the present invention. FIG. 3 depicts the specific processing of the particular DNA sequence 10 shown in FIG. 1. It is to be appreciated that the description of this specific method and processing only represents an exemplary embodiment of the present invention but is not to be taken to be in any way limiting or representing the only method. Suitable variants beyond this embodiment are also contemplated without departing from the invention.

As shown in FIGS. 2 and 3, the computer-implemented method of performing analysis of DNA sequences includes providing a compact representation of a raw DNA sequence 20a taken from a sample as an initial step 20. It should be appreciated that the invention is not limited to just DNA sequencing but can also be applied to any set of data represented by a collection of characters. The initial DNA sequence may be upper case, lower case or mixed case, but consistent case, preferably upper case, should be used to produce consistent results suitable for routine comparisons of DNA sequences. The method includes a computing step 22 of computing a digest of the computer-readable representation of the raw DNA sequence, where the resulting digest is in the form of a digest numerical value 22a. The step of computing the digest can include operating a hash function upon the raw DNA sequence to produce the digest numerical value. In one exemplary embodiment, the hash operation can be computed using a secure hash algorithm. (SHA) such as SHA-256, to output a digest numerical value 22a in the base-16 (hexadecimal) numeral system. However, the hash operation can also be computed using any other suitable algorithm such as cyclic redundancy check (CRC-32)

Generally speaking, the step of computing a digest of a raw DNA sequence can be performed by any suitable algorithm that is deterministic and maps distinct input I)NA sequences to a distinct digest numerical output value 22a in any positional number system, where it is understood that such positional number systems have a base number or radix indicative of the number of different values in each digit. For example, the common decimal system is base-10, where 10 is the radix, the hexadecimal system is base-16 and has a radix of 16, binary has a radix of 2, etc. In any event, in the embodiments of the present invention, the computed digest numerical value 22a is in a first positional numeral system having a first radix.

A converting step 24 is preformed of converting the digest numerical value 22a into a converted numerical value 24a. In the exemplary embodiment, the digest is converted to a base-26 number (i.e., having a radix of 26). For example, the base-16 digest numerical value 22a of the SHA-256 digest is converted to a base-26 number. The base-26 numerical system is selected for this embodiment in order to correspond to the English language alphabet and to thereby produce a desired end product. However, it is to be understood and appreciated that any suitable positional number system can be selected to correspond to the alphabet of any desired language, or alternatively, any set or subset of characters (e.g., Unicode characters) without departing from the invention. In any event, in the embodiments of the present invention, the computed converted numerical value 24a is in a second positional numeral system having a second radix.

The converted numerical value 24a is a number in which the significant digits are represented by a string of alphanumeric characters. However, for the purpose of the present method, an associating step 26 is performed of associating each significant digit of the converted numerical value 24a with a respective alphanumeric character of an encoding system. In this manner, the actual numerical value of each of the significant digits of the converted numerical value 24a are instead treated as characters 26a in an alphabet. In the exemplary embodiment, the characters 26a are ASCII English language alphanumerical characters, though they can alternatively be represented by alphanumeric characters in any other language. For example, in the illustrated example of FIG. 3, the second and third significant digits represented by “c” and “o” are treated as English language alphabet characters rather than the numerical values represented by the respective base-26 digits of the converted numerical value 24a.

In the standard 128-character ASCII table, decimal number values between 0-47, 58-64, 91-96, and 123-127 are used for various computer functions, and to represent punctuation and typographical symbols. Decimal number values between 48-57 are used to depict standard decimal numbers. Decimal values between 65-90 depict upper-case English alphabetical characters, and values between 97-122 depict lower-case English alphabetical characters, as shown in the following ASCII Table (Table 1):

TABLE 1
ASCII TABLE

| Dec | Char | Dec | Char | Dec | Char |
|---|---|---|---|---|---|
| 48 | 0 | 65 | A | 97 | a |
| 49 | 1 | 66 | B | 98 | b |
| 50 | 2 | 67 | C | 99 | c |
| 51 | 3 | 68 | D | 100 | d |
| 52 | 4 | 69 | E | 101 | e |
| 53 | 5 | 70 | F | 102 | f |
| 54 | 6 | 71 | G | 103 | g |
| 55 | 7 | 72 | H | 104 | h |
| 56 | 8 | 73 | I | 105 | i |
| 57 | 9 | 74 | J | 106 | j |
| | | 75 | K | 107 | k |
| | | 76 | L | 108 | l |
| | | 77 | M | 109 | m |
| | | 78 | N | 110 | n |
| | | 79 | O | 111 | o |
| | | 80 | P | 112 | p |
| | | 81 | Q | 113 | q |
| | | 82 | R | 114 | r |
| | | 83 | S | 115 | s |
| | | 84 | T | 116 | t |
| | | 85 | U | 117 | u |
| | | 86 | V | 118 | v |
| | | 87 | W | 119 | w |
| | | 88 | X | 120 | x |
| | | 89 | Y | 121 | y |
| | | 90 | Z | 122 | z |

In the associating step 26 according to the exemplary embodiment, each of the alphanumerical characters 26a are associated with ASCII characters according to the above ASCII Table.

For the purpose of arriving at a desired end product, a capitalizing step 28 is performed of capitalizing the lower-case ASCII alphanumeric characters represented by the significant digits into upper-case ASCII alphanumeric characters. In this manner, the second and third base-26 significant digits of the converted numerical value 24a represented by “c,” and “o” are capitalized into “C,” and “O” to produce a capitalized string of letters 28a. This step 26 is performed to reduce the number of ASCII characters and limit the values of the alphanumerical characters 26a to the ASCII decimal ranges of 48-57 and 65-90, which simplifies the subsequent processing.

The capitalizing step 28 is optional and not required for the generalized method, but the inventors have found this step 28 to be a practical offset to enable the end product to fall within a desired range of ASCII characters, resulting in a useful compact nomenclature outputted from the present computer-implemented method. However, it is to be understood and appreciated that any other suitable encoding system besides ASCII can be alternatively selected (e.g., Unicode) without departing from the invention. In such an instance, any other such suitable conversion besides capitalization and any resulting offsets can be alternatively performed in order to obtain a desired output in the suitable alternative encoding system.

A translating step 30 is performed of translating each of the respective ASCII alphanumeric characters 28a into their corresponding unique decimal number values, as shown in the ASCII Table above. As shown on the ASCII Table, each ASCII character is associated with a unique decimal number. For example, referring to the capitalized string of ASCII alphanumerical characters 28a, the ASCII character “C” is associated with the decimal number “67,” the ASCII character “3” is associated with the decimal number “51,” and so forth. In this manner, having previously translated the base-26 significant digits of the converted numerical value 24a into alphanumerical characters, the capitalized string of ASCII alphanumerical characters 28a is again translated back into unique decimal number values for subsequent processing.

Subsequent processing steps can be performed on the unique decimal number values to produce an end product represented by a code formed of a string of respective alphabetic characters. The resulting code has the desired number of characters, so that the code represents a compact nomenclature of the raw DNA sequence 20.

The subsequent processing steps can include a pair of selective addition steps 32 that are performed by 1) adding a value of ten to each of the unique decimal number values in the array 30a that represent ASCII alphabetic characters to produce first outputs, and 2) adding a value of seventeen to each of the unique decimal number values in the array 30a that represent ASCII number characters to produce second outputs. These respective first and second outputs are incorporated into respective locations of a modified array 32a composed of modified decimal number values. The offset values used are specific to converting ASCII values to capital English letters. Other offset values may be used for other alphabets or character sets.

The selective addition steps 32 are also optional steps not required by the generalized method, but the inventors have again found it practical to perform these steps 32 to provide offsets for producing first and second outputs that fall within desired decimal number ranges corresponding to ASCII characters to produce a useful compact nomenclature from the present computer-implemented method. Any similar additions or other mathematical operations could alternatively be performed as offsets in order to produce a desired end result from a suitable alternative encoding system (e.g., Unicode).

The subsequent processing steps can also include a converting step 34 of converting each of the modified decimal number values in the modified array 32a into a respective code 34a of ASCII alphabetic characters. This code 34a of ASCII alphabetic characters represents the encoded nomenclature of the raw DNA sequence 20.

As particularly shown in FIG. 3, the step 36 includes ordering the unique decimal number values in a transposed, reverse order from that of the capitalized string of ASCII alphanumerical characters 34a represented by the respective significant digits of the converted numerical value 24a. This results in a reversed, transposed array 36a of unique decimal number values. In this operation, the least significant digit represented in the capitalized string of ASCII alphanumerical characters 34a is now in the first place in the array 36a, and the most significant digit represented in the capitalized string of ASCII alphanumerical characters 34a is now in the last place in the array 36a, with all the other unique decimal number values being similarly transposed compared to the respective significant digits of the capitalized string of ASCII alphanumerical characters 34a.

The code 36a is compared with a second code representing a compact nomenclature of a second raw DNA sequence taken from a second DNA sample. This can then be used to determine whether a match exists between the code 36a and the second code, resulting in an identification of a candidate. For example, in forensic analysis of DNA sequences, the code 36a can represent a DNA sample taken from a crime scene while the second code can represent a second DNA sample can be taken directly from a suspect. In this manner, the two codes can be quickly and easily compared to determine whether a match exists, resulting in an identification of the suspect, locating this individual at the crime scene. Conversely, the code 36a taken from one DNA sequence appearing in a DNA sample, for example a crime scene sample, can be compared to the code 36a taken from a second DNA sequence appealing in the same DNA sample. The presence of multiple distinct codes within a sample indicates multiple distinct DNA sequences within the sample. These sequences may be true alleles from contributors to the sample, or artifactual sequences arising from DNA sequencing errors.

Moreover, the string of the code 36a represents a unique code for any specific DNA sequence within specific forensic contexts. In most instances, only the first two characters in the code 36a are required in forensic analysis to identify all distinct DNA sequences within a. forensic locus yielding the compact yet discriminating nomenclature 38a. The first two characters of a base-26 code cover 676 combinations. STR loci commonly used in forensic analysis exhibit fewer than 300 different distinct DNA sequences or alleles. For a listing of all known forensic locus alleles, see Gettings K B et al., “STRSeq: A catalog of sequence diversity at human identification Short Tandem Repeat loci.”. Forensic Sci Int Genet, 2017 Nov; 31:111-117.

In addition to true alleles from contributors to DNA samples, artifactual sequences derived from sequencing or PCR error may be present. Depending upon the sequencing method, the number of artifactual sequences may be large, thereby creating a large number of distinct sequences associated with any given forensic locus and thereby increasing the likelihood that using only the first two characters of the code will produce a “collision” where two distinct DNA sequences are mapped to the same 2-character code. In the event of a collision, it is found to be sufficient to allocate one or more additional characters to resolve the collision. Allocating three code characters of a base-26 code provides for 17,576 combinations; and allocating four code characters provides for 456,976 combinations.

In this “dynamic allocation” as many digits as necessary can be allocated to avoid collisions in a given analysis context. For example, analysis of forensic DNA profiles may involve less than 300 DNA sequences and relatively few digits will be required to discriminate all 300 sequences. Other scenarios may involve thousands or millions of sequences, and more digits will be required to avoid collisions when discriminating (i.e. uniquely labeling) sequences. In this manner, the compact nomenclature of the present invention simplifies forensic analysis and greatly improves accuracy.

The above-mentioned steps of the present method proceed sequentially, with the output of each step providing the input to the next step. In total, the method takes raw DNA sequences of any length or level of complexity as input; and produces a short letter code that represents that sequence as output. The original sequence could optionally be applied to RNA or protein.

As mentioned hereinabove, the present method can be practiced using a range of digest algorithms that produce fixed-length results. This range of algorithms include cryptologic hash algorithms such as SHA-1 and SHA-256 and cyclic redundancy check (CRC) algorithms such as CRC-32 or CRC-64. The method could also express compact codes using letters other than capital English letters by using respective alternative offsets selected to produce a desired result when converting digest numbers to final code characters.

The present method is especially useful in reducing errors typically encountered in a mixed sample, where multiple individuals have left DNA at a crime scene. The present method can be quickly performed on sequencer reads from a mixed DNA sample in search of a “signal” representing the DNA of a person of interest or other human candidate. In such instances, perhaps the crime scene DNA from blood of a victim in a mixed crime scene stain may provide a larger signal than crime scene DNA from a perpetrator. The present method provides superior results in discriminating differential DNA sequences from multiple individuals in mixed DNA crime scene samples. The “low signal” DNA sequences of the suspect can be clearly labeled and discriminated from the DNA sequences contributed by the victim or other persons at the crime scene.

The present method can be combined with a method to manage or classify DNA sequence artifacts. This extended method facilitates the use of Next Generation Sequencing (NGS) technology with mixed DNA analysis by binary methods or by Probabilistic Genotyping (PG) technology. One example is in forensic DNA analysis of short tandem repeat (STR) markers using targeted sequencing or NGS preceded by polymerase chain reaction (aka PCR-NGS methods). This is useful in detecting stutter artifacts that are produced in the PCR stage of the PCR-NGS method wherein PCR amplicons (DNA strands) are produced that have one less or one more repeated motif in the STR sequence. The present invention produces a simple way to link a given stutter artifact with the “parent” STR. marker through a combination of codes.

As an example, a parent STR marker may contain 6 STR motifs and be encoded using the left-most two digits or the two least significant digits of the code 36a produced by the method of the invention. Those two letters may be “AG.” A stutter artifact can be located at the 6-repeat STR marker and may contain only 5 repeat units and so the present method may produce a very different code 36a beginning with “RS.” The stutter artifact can be visually linked to the parent through a combination of codes such as “AG.RS,” with a period or “dot” symbol indicating a relationship.

Another type of artifact observed in PCR-NGS methods is called base substitution error, which is a type of error commonly observed in the NGS stage of PCR-NGS methods, but may also occur in the PCR stage where the phenomenon is commonly referred to as base misincorporation error, An example substitution error is the replacement of a cytosine (“C”) nucleotide at a specific position in the parent DNA molecule with a thymine (“T”) nucleotide. Whereas the sequence of the parent DNA molecule may be encoded as “RN” and the sequence of the artifactual molecule may be encoded as “WK.” The relationship of the artifactual sequence to the parent sequence can be represented by “RN”WK, with a grave accent symbol indicating a relationship.

Another type of artifact observed in PCR-NGS methods is called insertion and deletion error. Insertion and deletion errors refer to the erroneous insertion or deletion of one or more nucleotides in sequencer reads, This error may be a physical phenomenon where the DNA polymerase inserts to deletes one or more nucleotides during PCR amplification, or it may be a bioinformatic error where the sequencer instrument erroneously inserts or deletes one or nucleotides in the sequencer read which were not represented in the physical DNA strand.

The computer-implemented method of the present invention could be used in software user interfaces where DNA sequence information must be displayed, but where displaying the entire raw DNA sequence renders the display too crowded or complex, In applications where changes in DNA sequence can be detected, this method provides a way to quickly see differences without having to compare the original DNA sequences, by string matching or by alignment for example, as such techniques are known in the art. Since different DNA sequences produce different compact codes by this method, human operators can easily identify different sequences by simply looking at the codes.

Additionally, the computer-implemented method of the present invention can be used to encode any string of characters or numbers. These include DNA, RNA and protein sequence strings, In particular, one application of the method is in deconvolution and interpretation of mixed DNA samples commonly found at crime scenes which contain DNA from two or more persons. Mixture analysis software such as ‘probabilistic genotyping’ software performs this analysis using only the length feature of DNA sequences. The length feature is easily encoded to a simple numbering system that identifies both alleles (e.g. from STR markers) and stutter artifacts of those alleles. Further, the mixture analysis software must be able to use this coding system to associate stutter artifacts with the ‘parent’ alleles from which they were derived. New “next generation sequencing” (NGS) technology is now beginning to be used in forensic DNA analysis, and this method provides a way to encode alleles and stutter and to provide a way to associate stutter and alleles, thereby enabling the application of probabilistic genotyping software to NGS data.

The computer-implemented method of the present invention can also facilitate data processing and retrieval. For example, the present compact nomenclature would enable rapid searching through national and international databases to locate matches to the codes produced by the present method, greatly enhancing accuracy and efficiency. Also, the present method enables rapid searching for chromosome features such as SNPs, which would facilitate the accurate location of samples in various research establishments.

The present method can rapidly detect rare and unanticipated alleles in forensic samples. For example, a DNA sequence containing an STR locus and exhibiting a particular STR allele, say an 18-repeat STR. allele, at that locus may also exhibit a minor or rare allele at a SNP locus elsewhere in the DNA sequence under analysis. The presence of the rare SNP allele will cause the method to generate a code distinct from the code normally associated with the analyzed DNA segment containing the 18-repeat STR allele. Whereas the presence of a single nucleotide change may not be readily apparent from visual inspection of the raw DNA sequence, the presence of the distinct method code will provide a rapid and easy way to detect the presence of the rare variant.

The following examples are provided herewith to illustrate the utility and benefits of the method and system of the present invention.

### EXAMPLE 1

### A 107-Ncleotide Sequence (SEQ ID NO. 1)

TATTTAGTGAGATTAAAAAAAACTATCAATCTGTCTATCTATCTATC
TATCTATCTATCTATCTATCTATCTATCTATCTATCTATCGTTAGTT
CGTTCTAAACTAT

that includes an allele of the forensic STIR locus D75820 with 13 tandem repeats of the [TATC] motif as well as 35 nucleotides of the upstream flank including the bi-allelic SNP locus rs7789995 exhibiting the “T” allele; and 20 nucleotides in the downstream flank including the bi-allelic SNP locus rs16887642 exhibiting the “G” allele was processed by the method using the SHA-256 hash function. In this fragment, the SNP locus rs7789995 is located 14 nucleotides from the left (upstream) end of the fragment and the SNP locus rs16887642 is located 12 nucleotides from the right (downstream) end of the fragment.

The nomenclature generated by the method of the present invention is:

QWIYGSEAUXYNFOOWNICHLOZAASSUBLUFONLCUKWELUASCOO
TBSVEEPD

The 2-letter nomenclature of the method of the present invention is QW, which can be used to compactly label the sequence for comparison or other purposes.

The equivalentdescription of this DNA sequence using them ethod of Parsons et al. is:
D7S840[CE13]-Chr7-CiRCh38 84160191-84160297 [TATC]13
   which is too verbose for use in many computer interface situations, and it too verbose for use in verbal communication. The equivalent description of this DNA sequence using the method of Just et al. is: 13_13, which does not capture the full sequence information especially the sequence variation present at the SNP loci. Thus, this example illustrates the practical utility, advantages and benefits as compared with prior art methods.

### EXAMPLE 2

### The 107-nucleotide sequence (SEQ ID NO. 2)

TATTTAGTGAGATAAAAAAAAACTATCAATCTGTCTATCTATCTATC
TATCTATCTATCTATCTATCTATCTATCTATCTATCTATCGTTAGTT
CATTCTAAACTAT

that includes an allele of the forensic STR locus D7S820 with 13 tandem repeats of the [TATC] motif as well as 35 nucleotides of the upstream flank including the bi-allelic SNP locus rs7789995 exhibiting the “A” allele; and 20 nucleotides in the downstream flank including the bi-al lelic SNP locus rs16887642 exhibiting the “A” allele was processed by the method using the SHA-256 hash function. In this fragment, the SNP locus rs7789995 is located 14 nucleotides from the left (upstream) end of the fragment and the SNP locus rs16887642 is located 12 nucleotides from the right (downstream) end of the fragment.

The label generated by the method of the present invention is

JJELCSTLTACAOZICVBFBCCGLWQPOHHWZBBDFJKPUGSWWLXV
NPOSDQWB

The 2-letter label of the method of the present invention is JJ.

The equivalent description of this DNA sequence using the method of Parsons et al. is:
D7S840[CE13]-Chr7-GRCh38 84160191-84160297 [TATC]13 84160204-A; 84160286-A
   The equivalent description of this DNA sequence using the method of Just et al. is: 13_13. The present method discriminates the two sequences of Examples 1 and 2 using just two letters, whereas the method of Parsons et al., requires 51 or 74 characters. The method of Just et al. does not discriminate the sequences. Thus, this example further illustrates the practical utility, advantages and benefits as compared with prior art methods.

### EXAMPLE 3

This example focused on the analysis of pure standard DNA material 2800M sequenced according to Sharma et al., haps://doi.org/10.1371/journal.pone.0187932. (See Promega corporatiorr for known genotypes of standard material 2800M at https://www.promega.corn.)

The sample is known to be heterozygous at the D8S1179 locus and thus the analyzed data includes two alleles. The raw fastq-formatted data file was analyzed using MixtureAce software (NicheVision, LLC) where the analyzed sequence ranged from Chr8: 124894865-124894921 of the GRCh38 genomic assembly. Sequencer reads are associated with loci through the recognition of the PCR primer binding sites in the PCR amplicon sequences.

The first 20 nucleotides of the upstream and downstream PCR binding sites used by the ForenSeq PCR kit (Verogen, Inc.) for the D8S1179 locus are:

(GenBank top strand orientation)
(SEQ ID NO. 3)
ATTTCATGTGTACATTCGTA;
and
(GenBank bottom strand orientation)
(SEQ ID NO. 4)
TGTAGATTATTTTCACTGTG
respectively.

This segment includes no upstream nucleotides but 5 downstream nucleotides, making the true allelic sequences 61 and 65 nucleotides in length. All distinct sequences attributable to the D8S1179 locus present at a read count intensity of greater than or equal o 10 reads were tabulated as shown in Table 2:

TABLE 2

| | | Method | | | |
|---|---|---|---|---|---|
| Read | Typing | Short | Method Long | STR Sequence in | DNA Sequence |
| Count | Call | Label | Label | Bracketed Format | |
| 2,764 | Allele | YO | YOWLYFTTZTMMUMZGRL | [TCTA]1[TCTG]1 | TCTATCTGTCTATCTATCTATCT |
| | | | LQUJWEJJYJRCVQFXFJ | [TCTA]12 | ATCTATCTATCTATCTATCTATC |
| | | | TKQXTIENRNNMJELRUM | | TATCTATCTATTCCC |
| | | | | | (SEQ ID NO. 5) |
| 346 | N-1 | YO.MR | MRCXEGQXVIMDHLVJET | [TCTA]1[TCTG]1 | TCTATCTGTCTATCTATCTATCT |
| | Stutter | | OJPQECVKLDKVTUUHVT | [TCTA]11 | ATCTATCTATCTATCTATCTATC |
| | | | NUTPGTUHIUZDUQTHPU | | TATCTATTCCC |
| | | | D | | (SEQ ID NO. 6) |
| 30 | N-2 | YO.KL | KLQLNOAXWJXDAFVGHG | [TCTA]1[TCTG]1 | TCTATCTGTCTATCTATCTATCT |
| | Stutter | | AQVASOBTXULWGFKOJA | [TCTA]10 | ATCTATCTATCTATCTATCTATC |
| | | | AXKOFBVPEQOICWVOJP | | TATTCCC |
| | | | B | | (SEQ ID NO. 7) |
| 2,977 | Allele | HI | HIMZRKENYBLTUHUPXJ | [TCTA]2[TCTG]1 | TCTATCTATCTGTCTATCTATCT |
| | | | EQKAJFQMTGJXYUJNLW | [TCTA]12 | ATCTATCTATCTATCTATCTATC |
| | | | KTZARQYYOAKTMRMUVY | | TATCTATCTATCTATTCCC |
| | | | | | (SEQ ID NO. 8) |
| 383 | N-1 | HI.BK | BKSMZCWYLGGZLNOAJL | [TCTA]2[TCTG]1 | TCTATCTATCTGTCTATCTATCT |
| | Stutter | | VZAOXLUPJEWDTCLPVH | [TCTA]11 | ATCTATCTATCTATCTATCTATC |
| | | | RYCFPQIEIDZBJCAHMC | | TATCTATCTATTCCC |
| | | | | | (SEQ ID NO. 9) |
| 61 | N-2 | HI.QX | QXTCZPVDQPOBUPUTCS | [TCTA]2[TCTG]1 | TCTATCTATCTGTCTATCTATCT |
| | Stutter | | DGGSBEVVUJKRJJFSFK | [TCTA]10 | ATCTATCTATCTATCTATCTATC |
| | | | FZFQMZTUDVHHTQSIBD | | TATCTATTCCC |
| | | | B | | (SEQ ID NO. 10) |
| 13 | N + 1 | HI.JF | JFHMLLQIKTUEKATASN | [TCTA]2[TCTG]1 | TCTATCTATCTGTCTATCTATCT |
| | Stutter | | DHHFCMIQNEWUUXKITT | [TCTA]13 | ATCTATCTATCTATCTATCTATC |
| | | | QDGPFDXTGTHECRARYB | | TATCTATCTATCTATCTATTCCC |
| | | | D | | (SEQ ID NO. 11) |
| 18 | Sequence | HI′CP | CPZPUWWGOUKHDZUNJM | [TCTA]2[TCTG]1 | TCTATCTATCTGTCTATCTATCT |
| | Error | | ZCHCMOLVARXJYBEJVF | [TCTA]12 | ATCTATCTATCTATCTATCTATC |
| | | | UEHVRHYOMCCNMHPPRT | | TATCTATCTATCTATTCCT |
| | | | | | (SEQ ID NO. 12) |

The two true allelic sequences exhibit 14 and 15 tandemly repeated tetramer motifs respectively. These two alleles were present in the data at 2,764 and 2,977 sequencer reads respectively. The allelic sequences are labeled by the method as YO and HI respectively. In addition to the 2 true allelic sequences, 6 artifactual sequences were observed. All 8 distinct sequences associated with the D8S1179 locus are discriminated using two letters of the method label. Furthermore, artifacts can be linked to the alleles from which they derived using standard knowledge of the patterns and intensity of artifact identification. Five of the artifacts are expected stutter products of PCR and their association with their ‘parent’ allele can be indicated using a period character in ‘parent.artiface’ notation, or any other convenient notation.

For a description of stutter artifact calling see Butler, J. M., Fundamentals of Forensic DNA Typing 2010 ISBN 978-0-12-374999-4. One artifactual sequence is observed to differ from its assigned parent true allele by a single nucleotide error in the right-most nucleotide in the read sequence. This artifact is likely due to one or the other of known error sources: PCR misincorporation error, or sequencing error. Sequence errors can be recognized by the closeness of alignment to the parent sequence. The association of the artifactual sequence labeled ‘CP’ with the true allele sequence labeled ‘HI’ using a grave character in ‘parent’ error’ notation, or any other convenient notation. Sequence errors can be stochastic in sequencing systems and therefore can produce error sequences that are unexpected, or not previously observed. The method is robust to distinctly labeling any sequence string regardless of prior observation.

### EXAMPLE 4

This example focuses on analysis of a 3:1 mixture (mass of DNA basis) of two standard DNA materials: 2800M and 2391c component A. See NIST certificate of analysis for known genotypes of standard reference material 2391c component A at https://www.nist.gov/srm. Sequencing and data analysis were performed as described in Example 3 above. Both standard materials are known to be heterozygous at locus D8S1179 with all four alleles exhibiting distinct sequences as shown in Table 3:

TABLE 3

| | | Method | | | |
|---|---|---|---|---|---|
| | Typing | Short | Method | Bracketed | |
| Count | Call | Label | Long Label | STR Sequence | DNA Sequence |
| 605 | Allele | YO | YOWLYFTTZTMMUMZGRL | [TCTA]1[TCTG]1 | TCTATCTGTCTATCTATCTATCT |
| | | | LQUJWEJJYJRCVQFXFJ | [TCTA]12 | ATCTATCTATCTATCTATCTATC |
| | | | TKQXTIENRNNMJELRUM | | TATCTATCTATTCCC |
| | | | | | (SEQ ID NO. 5) |
| 77 | N-1 | YO.MR | MRCXEGQXVIMDHLVJET | [TCTA]1[TCTG]1 | TCTATCTGTCTATCTATCTATCT |
| | Stutter | | OJPQECVKLDKVTUUHVT | [TCTA]11 | ATCTATCTATCTATCTATCTATC |
| | | | NUTPGTUHIUZDUQTHPU | | TATCTATTCCC |
| | | | D | | (SEQ ID NO. 13) |
| 500 | Allele | HI | HIMZRKENYBLTUHUPXJ | [TCTA]2[TCTG]1 | TCTATCTATCTGTCTATCTATCT |
| | | | EQKAJFQMTGJXYUJNLW | [TCTA]12 | ATCTATCTATCTATCTATCTATC |
| | | | KTZARQYYOAKTMRMUVY | | TATCTATCTATCTATTCCC |
| | | | | | (SEQ ID NO. 8) |
| 26 | N-2 | HI.QX | QXTCZPVDQPOBUPUTCS | [TCTA]2[TCTG]1 | TCTATCTATCTGTCTATCTATCT |
| | Stutter | | DGGSBEVVUJKRJJFSFK | [TCTA]10 | ATCTATCTATCTATCTATCTATC |
| | | | FZFQMZTUDVHHTQSIBD | | TATCTATTCCC |
| | | | B | | (SEQ ID NO. 10) |
| 214 | Allele | BK | BKSMZCWYLGGZLNLOAJ | [TCTA]2[TCTG]1 | TCTATCTATCTGTCTATCTATCT |
| | | | LVZAOXLUPJEWDTCLPV | [TCTA]11 | ATCTATCTATCTATCTATCTATC |
| | | | HRYCFPQIEIDZBJCAHM | | TATCTATCTATTCCC |
| | | | C | | (SEQ ID NO. 9) |
| 147 | Allele | WH | WHXBXOBNGIIDVYWZWM | [TCTA]13 | TCTATCTATCTATCTATCTATCT |
| | | | MONQNQUZOQPONDCJBS | | ATCTATCTATCTATCTATCTATC |
| | | | OULVKQBONOKGYEFESG | | TATCTATTCCC |
| | | | | | (SEQ ID NO. 14) |
| 20 | N-1 | WH.CM | CMDXWAXEMNMKKRAGCS | [TCTA]12 | TCTATCTATCTATCTATCTATCT |
| | Stutter | | ZGOISVBORLQIAWXYGQ | | ATCTATCTATCTATCTATCTATC |
| | | | FGFPXHVFPWHHTQPQVC | | TATTCCC |
| | | | | | (SEQ ID NO. 15) |

The method generated distinct 2-letter labels for each of the 4 allelic and 3 artifactual sequences. Additionally, the 2-letter labels can be associated as described in Example 3 to indicate which artifact is associated with which true allele. This explicit attribution enabled by the method is important in mixed DNA analysis in law enforcement scenarios where it is critical that artifacts are assigned as such and therefore discriminated from the presence of additional true alleles in the mixture from additional contributors to the mixture. In this example, the sequence QX is attributed as N-2 stutter of the true allele HI, and this is indicated by the ‘HI.QX’ convention. Here the term N-2 refers to a stutter product exhibiting 2 fewer motifs relative to that of the parent allele that exhibits N tandemly repeated motifs. The QX sequence could also plausibly be attributed as N-1 stutter of the true allele labeled BK (i.e. BK,QX); or it could be attributed as arising from a combination of these sources.

The labels generated by the present method provide a means of unequivocally describing the attributions of observed sequences in DNA mixtures to categories such as true alleles from contributors to the mixture, or artifacts of true alleles. The nomenclature is independent of the method used to make attributions can be manual analysis, software analysis, or a combination of these.

### EXAMPLE 5

This example focuses on the analysis of microhaplotype markers. Microhaplotype markers are a marker class developed for use in forensic analysis and tend to consist of two or more SNP markers close enough together to be amplified in the same PCR amplicon, and therefore will be in a phase-known state. (For details on microhaplotype markers see Kidd, K. K., et al., Forensic Sci. Int. Genet. 12 (2014) 215-224. doi:10.10161 Isigen.2014.06.014.)

Forensic microhap markers are but one example of the class of haplotype markers, and the principles applied to microhaplotype markers apply to haplotypes generally including mitochondrial haplotypes and haplotypes that include different marker types such as SNP, DIP or SIR markers in combinations. One microhaplotype marker is mh02KK-003 which consists of three component SNP loci: rs260694 (a G/T SNP), rs11123719 (a T/C SNP), and rs11691107 (a C/T SNP), where all nucleotide letters refer to GenBank top strand. The genomic locations of these SNPs in the GRCh38 assembly are: chr2:108,969,857; chr2:108,969,915; and chr2:108,969,981. The DNA sequence of the GRCh38 reference across these positions is:

(SEQ ID NO. 16)
GATCCAAAAAGGGGTGAAAGAATCACTGAGTTAGAGAAGGCTTCAGG
AGAATCCAGAGTTCAATCTGGGTCATAAGAACATACAACTCAGATTT
CTTTAAACACAGTTAAAAGTGGGGAAATTGC

There are exactly 8 possible observable haplotypes for this microhaplotype assuming each SNP is biallelic, where short-hand descriptions of the haplotypes is based on the nucleotide present at each of the component SNP locations: GTC, GTT, GCC, GCT, TTC, TTT, TCC, and TCT (all GenBank top strand). See Table 4 for a foil description of these microhaps:

TABLE 4

| | | Method | | | |
|---|---|---|---|---|---|
| | | Short | | SNP | |
| Row | Item | Label | Method Long Label | States | DNA Sequence |
| 1 | Possible | YL | YLHINODJDZUREKTAUNLJML | GTC | GATCCAAAAAGGGGTGAAAGAATCACTGAGTTAGAGAAG |
| | mh02KK- | | YELDIVYUATVEJPMTMNZHLD | | GCTTCAGGAGAATCCAGAGTTCAATCTGGGTCATAAGAA |
| | 003 | | DHSRQAPXFHC | | CATACAACTCAGATTTCTTTAAACACAGTTAAAAGTGGG |
| | Allele | | | | GAAATTGC (SEQ ID NO. 16) |
| 2 | Possible | GE | GEPRWIRYWZOPACPPRSURDI | GTT | GATCCAAAAAGGGGTGAAAGAATCACTGAGTTAGAGAAG |
| | mh02KK- | | LGZMVCSUMWXPZSQGAATEIW | | GCTTCAGGAGAATCCAGAGTTCAATCTGGGTCATAAGAA |
| | 003 | | YTSARUKXHC | | CATACAACTCAGATTTCTTTAAACACAGTTAAAAGTGGG |
| | Allele | | | | GAAATTGT (SEQ ID NO. 17) |
| 3 | Possible | PN | PNHUVYFZIRZAIPSCJTRXPQ | GCC | GATCCAAAAAGGGGTGAAAGAATCACTGAGTTAGAGAAG |
| | mh02KK- | | QLDZLKJIMNRQDVZGUIKHOC | | GCTTCAGGAGAATCCAGAGCTCAATCTGGGTCATAAGAA |
| | 003 | | ZHONILAHEC | | CATACAACTCAGATTTCTTTAAACACAGTTAAAAGTGGG |
| | Allele | | | | GAAATTGC (SEQ ID NO. 18) |
| 4 | Possible | AN | ANNULRDUWZWDLGENIZCSLL | GCT | GATCCAAAAAGGGGTGAAAGAATCACTGAGTTAGAGAAG |
| | mh02KK- | | HOHZHWQLCKUSZKBMZOQNMO | | GCTTCAGGAGAATCCAGAGCTCAATCTGGGTCATAAGAA |
| | 003 | | BIKNNEAFMKB | | CATACAACTCAGATTTCTTTAAACACAGTTAAAAGTGGG |
| | Allele | | | | GAAATTGT (SEQ ID NO. 19) |
| 5 | Possible | JQ | JQGHOQIZJQGMJAHZGSCUAP | TTC | TATCCAAAAAGGGGTGAAAGAATCACTGAGTTAGAGAAG |
| | mh02KK- | | SLFGHIJKRHPLYEXRKFKUXV | | GCTTCAGGAGAATCCAGAGTTCAATCTGGGTCATAAGAA |
| | 003 | | CPGKQDGASQB | | CATACAACTCAGATTTCTTTAAACACAGTTAAAAGTGGG |
| | Allele | | | | GAAATTGC (SEQ ID NO. 20) |
| 6 | Possible | QR | QRGEGSXYFYHONOWIHFVIHQ | TTT | TATCCAAAAAGGGGTGAAAGAATCACTGAGTTAGAGAAG |
| | mh02KK- | | MAUUAGDVKRQOQYXPWIRKEQ | | GCTTCAGGAGAATCCAGAGTTCAATCTGGGTCATAAGAA |
| | 003 | | MSZHONGWE | | CATACAACTCAGATTTCTTTAAACACAGTTAAAAGTGGG |
| | Allele | | | | GAAATTGT (SEQ ID NO. 21) |
| 7 | Possible | US | USOCFFRLHSISFJWAGJNLMO | TCC | TATCCAAAAAGGGGTGAAAGAATCACTGAGTTAGAGAAG |
| | mh02KK- | | ATYWFYUCOPAVOJTXDMBRNR | | GCTTCAGGAGAATCCAGAGCTCAATCTGGGTCATAAGAA |
| | 003 | | RGNHFDXAVME | | CATACAACTCAGATTTCTTTAAACACAGTTAAAAGTGGG |
| | Allele | | | | GAAATTGC (SEQ ID NO. 22) |
| 8 | Possible | RG | RGHISIFZPRAETNYNPEVWJL | TCT | TATCCAAAAAGGGGTGAAAGAATCACTGAGTTAGAGAAG |
| | mh02KK- | | NYWXESSGJXBSMRIMHTGGRF | | GCTTCAGGAGAATCCAGAGCTCAATCTGGGTCATAAGAA |
| | 003 | | WNAFDSOXFDC | | CATACAACTCAGATTTCTTTAAACACAGTTAAAAGTGGG |
| | Allele | | | | GAAATTGT (SEQ ID NO. 23) |
| 9 | Undefined | LG | LGXYBDSEFUHOHAQYULQKVT | GATC | GATCCAAAAAAGGGTGAAAGAATCACTGAGTTAGAGAAG |
| | Allele | | SJTIMVKUMPYLZFKBDRDSRA | | GCTTCAGGAGAATCCAGAGTTCAATCTGGGTCATAAGAA |
| | | | MAQZODUMTG | | CATACAACTCAGATTTCTTTAAACACAGTTAAAAGTGGG |
| | | | | | GAAATTGC (SEQ ID NO. 24) |
| 10 | Undefined | FO | FOEGHKXUIYQPEIRCVBUBIB | GATC | GATCCAAAAAGGGATGAAAGAATCACTGAGTTAGAGAAG |
| | Allele | | RGQIUKUYONTNHPQYULNPTW | | GCTTCAGGAGAATCCAGAGTTCAATCTGGGTCATAAGAA |
| | | | MCCYBAOTAED | | CATACAACTCAGATTTCTTTAAACACAGTTAAAAGTGGG |
| | | | | | GAAATTGC (SEQ ID NO. 25) |

(Table 4: Microhap mh02KK-003 is a 3-SNP microhap consisting of rs260694 (a G/T SNP), rs11123719 (a T/C SNP), and rs11691107 (a C/T SNP) where all nucleotide letters refer to GenBank top strand. The 8 possible component SNP states (aka alleles) for this microhap are listed in rows 1-8 and the variable positions are underlined. Labels produced by the present method are shown in the columns labeled ‘method short label’ and ‘method long label’. The short-hand descriptions of the SNP states are listed in the column labeled ‘SNP states’, and the raw sequences between GRCh38 positions chr2:108,969,857 and chr2:108,969,981 (inclusive) are listed in the column labeled ‘sequence’. Table 4 rows 9 and 10 display sequences that arise when either SNP rs563073581 or rs1265297626 exhibit the ‘A’ nucleotide, while the remainder of the sequence exhibits the GRCh38 reference sequence e the sequence in row 1). The variable positions of these two SNPs are underlined.)

The microhap nomenclature and the short-hand description of haplotypes is robust to the possible future discovery that one or more of the SNPs is tri- or tetra-allelic. However, the nomenclature and short-hand descriptions are not robust to the possible future discovery of an additional polymorphic position within the microhaplotype extent.

An additional 22 SNPs are cataloged within this region (dbSNP, build 151; https://www.ncbi.nlm.nih.gov/snp/), and any of these may be polymorphic in any given human population. Inclusion of a new polymorphic position requires re-defining the component SNPs of the mh02KK-003 microhap or establishment of a new microhap description. For laboratories conducting routine forensic analysis, this is inconvenient or impractical.

The present invention depends solely on the nucleotide sequence and is therefore robust to any nucleotide sequence change. For example, SNP rs563073581 (a G/A SNP) and rs1265297626 (a G/A SNP) are located at GRCh38 genomic positions chr2:108,969,867 and chr2:108,969,870 respectively and are therefore contained within the region described by microhap mh02KK-003. If the alternate nucleotide for one or the other SNP is observed to be present in an individual, then the microhap nomenclature will be invalidated, and the short-hand description of haplotypes will be identical as: ‘GATC’. Additional annotation is required to identify which SNP polymorphism accounted for the additional ‘A’ nucleotide in the shorthand. However, the nucleotide sequence will still be encodable by the present method. The distinct labels produced for these sequences are:

LGXYBDSEFUHOHAQYULQKVTSJTIMVKUMPYLZFKBDRDSRAMAQ
ZODUMTG
and
FOEGHKXUIYQPEIRCVBUBIBRGQIUKUYONTNHPQYULNPTWMCC
YBAOTAED

where the first 2 characters are sufficient to discriminate all 8 possible mh02KK-003 alleles and the 2 additional sequences. See Table 4 for a description of the nucleotide sequences and the corresponding method labels.

The embodiments have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

Having thus described the invention, it is now claimed:

What is claimed:

1. A computer-implemented method of performing analysis of DNA sequences, comprising:
providing a computer-readable representation of a raw DNA sequence taken from a first DNA sample;
computing a digest of the computer-readable representation of the raw DNA sequence, wherein the digest comprises a digest numerical value, wherein the digest numerical value is in a first positional numeral system having a first radix;
converting the digest numerical value into a converted numerical value, wherein the converted numerical value is in a second positional numeral system having a second radix, wherein the second radix is selected to produce an end product having a predetermined number of characters;
associating each significant digit of the converted numerical value with a respective alphanumeric character of an encoding system;
translating each of the respective alphanumeric characters into their corresponding unique numbers of the encoding system;
processing the corresponding unique numbers into the end product represented by a code of respective alphabetic characters, the code having the predetermined number of characters, wherein the code represents a compact nomenclature of the raw DNA sequence taken from the first DNA sample; and
comparing the code with a second code representing a compact nomenclature of a second raw DNA sequence taken from a second DNA sample to determine whether a match exists between the code and the second code, resulting in an identification of a candidate.

2. The computer-implemented method of claim 1, wherein the computing of the digest comprises operating a hash function upon the raw DNA sequence to produce the digest numerical value.

3. The computer-implemented method of claim 2, wherein the operating of the hash function includes selecting a hash function from SHA-256 or CRC-32.

4. The computer-implemented method of claim 1, wherein the converting comprises converting the digest numerical value from a first positional numeral system having a radix of 16, corresponding to a base-16 numeral system, into a corresponding converted numerical value in a second positional number system having a radix of 26, corresponding to a base-26 numeral system.

5. The computer-implemented method of claim I, wherein the encoding system is selected from ASCII and Unicode.

6. The computer-implemented method of claim 1, wherein the associating of each significant digit of the converted numerical value with the respective alphanumeric character further comprises capitalizing any lower-case letters of the significant digits into upper-case letters, and wherein the translating of each of the respective alphanumeric characters comprises translating the respective upper-case letters into their corresponding unique numbers of the encoding system.

7. The computer-implemented method of claim I, wherein the processing of the corresponding unique numbers into the end product further comprises ordering the unique numbers in a transposed, reverse order from that of the respective significant digits of the converted numerical value.

8. The computer-implemented method of claim 7, wherein the processing of the corresponding unique numbers into the end product further comprises:
adding a first number value to each of the unique numbers that represent alphabetic characters of the encoding system to produce first outputs;
adding a second number value to each of the unique numbers that represent number characters of the encoding system to produce second outputs; and
converting each of the first and second outputs into respective alphabetic characters of the encoding system, wherein the first and second number values are selected to produce alphabetic characters after converting, such that the end product comprises a string of alphabetic characters representing a compact nomenclature of the raw DNA sequence.

9. The computer-implemented method of claim 1, further comprising using the code of the end product in a forensic DNA analysis to match the raw DNA sequence with a second raw DNA sequence taken from a second DNA sample from a human candidate.

10. A computer-implemented method of performing forensic analysis of DNA sequences, comprising:
providing a computer-readable representation of a raw DNA sequence taken from a first DNA sample;
computing a digest of the computer-readable representation of the raw DNA sequence, wherein the digest comprises a digest numerical value. wherein the digest numerical value is in a base-16 numeral system;
converting the digest numerical value into a converted numerical value, wherein the converted numerical value is in a base-26 numeral system;
associating each significant digit of the converted numerical value with a respective ASCII alphanumeric character;
capitalizing any lower-case ASCII alphanumeric characters of the significant digits into upper-case ASCII alphanumeric characters;
translating each of the respective ASCII alphanumeric characters into their corresponding unique decimal number values;
ordering the unique decimal number values in a transposed, reverse order from that of the respective significant digits of the converted numerical value; and
adding a value of ten to each of the unique decimal number values that represent ASCII alphabetic characters to produce first outputs;
adding a value of seventeen to each of the unique decimal number values that represent ASCII number characters to produce second outputs;
converting each of the first and second outputs into a respective code of ASCII alphabetic characters representing a compact nomenclature of the raw DNA sequence taken from the first DNA sample; and
comparing the code with a second code representing a compact nomenclature of a second raw DNA sequence taken from a second DNA sample to determine whether a match exists between the code and the second code, resulting in an identification of a human candidate.


SEQUENCE LISTING (26 sequences)

SEQ ID NO: 1 - DNA, 107 bp, Homo sapiens
tatttagtgagattaaaaaaaactatcaatctgtctatctatctatctatctatctatctatctatctatctatctatctatctatcgttagttcgttctaaactat

SEQ ID NO: 2 - DNA, 107 bp, Homo sapiens
tatttagtgagataaaaaaaaactatcaatctgtctatctatctatctatctatctatctatctatctatctatctatctatctatcgttagttcattctaaactat

SEQ ID NO: 3 - DNA, 20 bp, Homo sapiens
atttcatgtgtacattcgta

SEQ ID NO: 4 - DNA, 20 bp, Homo sapiens
tgtagattattttcactgtg

SEQ ID NO: 5 - DNA, 61 bp, Homo sapiens
tctatctgtctatctatctatctatctatctatctatctatctatctatctatctattccc

SEQ ID NO: 6 - DNA, 57 bp, Homo sapiens
tctatctgtctatctatctatctatctatctatctatctatctatctatctattccc

SEQ ID NO: 7 - DNA, 53 bp, Homo sapiens
tctatctgtctatctatctatctatctatctatctatctatctatctattccc

SEQ ID NO: 8 - DNA, 65 bp, Homo sapiens
tctatctatctgtctatctatctatctatctatctatctatctatctatctatctatctattccc

SEQ ID NO: 9 - DNA, 61 bp, Homo sapiens
tctatctatctgtctatctatctatctatctatctatctatctatctatctatctattccc

SEQ ID NO: 10 - DNA, 57 bp, Homo sapiens
tctatctatctgtctatctatctatctatctatctatctatctatctatctattccc

SEQ ID NO: 11 - DNA, 69 bp, Homo sapiens
tctatctatctgtctatctatctatctatctatctatctatctatctatctatctatctatctattccc

SEQ ID NO: 12 - DNA, 65 bp, Homo sapiens
tctatctatctgtctatctatctatctatctatctatctatctatctatctatctatctattcct

SEQ ID NO: 13 - DNA, 57 bp, Homo sapiens; identical to SEQ ID NO: 6

SEQ ID NO: 14 - DNA, 57 bp, Homo sapiens
tctatctatctatctatctatctatctatctatctatctatctatctatctattccc

SEQ ID NO: 15 - DNA, 53 bp, Homo sapiens
tctatctatctatctatctatctatctatctatctatctatctatctattccc

SEQ ID NO: 16 - DNA, 125 bp, Homo sapiens
gatccaaaaaggggtgaaagaatcactgagttagagaaggcttcaggagaatccagagttcaatctgggtcataagaacatacaactcagatttctttaaacacagttaaaagtggggaaattgc

SEQ ID NO: 17 - DNA, 125 bp, Homo sapiens
gatccaaaaaggggtgaaagaatcactgagttagagaaggcttcaggagaatccagagttcaatctgggtcataagaacatacaactcagatttctttaaacacagttaaaagtggggaaattgt

SEQ ID NO: 18 - DNA, 125 bp, Homo sapiens
gatccaaaaaggggtgaaagaatcactgagttagagaaggcttcaggagaatccagagctcaatctgggtcataagaacatacaactcagatttctttaaacacagttaaaagtggggaaattgc

SEQ ID NO: 19 - DNA, 125 bp, Homo sapiens
gatccaaaaaggggtgaaagaatcactgagttagagaaggcttcaggagaatccagagctcaatctgggtcataagaacatacaactcagatttctttaaacacagttaaaagtggggaaattgt

SEQ ID NO: 20 - DNA, 125 bp, Homo sapiens
tatccaaaaaggggtgaaagaatcactgagttagagaaggcttcaggagaatccagagttcaatctgggtcataagaacatacaactcagatttctttaaacacagttaaaagtggggaaattgc

SEQ ID NO: 21 - DNA, 125 bp, Homo sapiens
tatccaaaaaggggtgaaagaatcactgagttagagaaggcttcaggagaatccagagttcaatctgggtcataagaacatacaactcagatttctttaaacacagttaaaagtggggaaattgt

SEQ ID NO: 22 - DNA, 125 bp, Homo sapiens
tatccaaaaaggggtgaaagaatcactgagttagagaaggcttcaggagaatccagagctcaatctgggtcataagaacatacaactcagatttctttaaacacagttaaaagtggggaaattgc

SEQ ID NO: 23 - DNA, 125 bp, Homo sapiens
tatccaaaaaggggtgaaagaatcactgagttagagaaggcttcaggagaatccagagctcaatctgggtcataagaacatacaactcagatttctttaaacacagttaaaagtggggaaattgt

SEQ ID NO: 24 - DNA, 125 bp, Homo sapiens
gatccaaaaaagggtgaaagaatcactgagttagagaaggcttcaggagaatccagagttcaatctgggtcataagaacatacaactcagatttctttaaacacagttaaaagtggggaaattgc

SEQ ID NO: 25 - DNA, 125 bp, Homo sapiens
gatccaaaaagggatgaaagaatcactgagttagagaaggcttcaggagaatccagagttcaatctgggtcataagaacatacaactcagatttctttaaacacagttaaaagtggggaaattgc

SEQ ID NO: 26 - DNA, 87 bp, Homo sapiens
gccaggaactgtggctcatctatgaaaacttctatctatctatctatctatctatctatctatctatctatctatcatatcataaca